(19) United States
(12) Patent Application Publication
(10) Pub. No.: US 2006/0067468 A1
(43) Pub. Date: Mar. 30, 2006

(54) Radiotherapy systems

(76) Inventor: Eike Rietzel, Darmstadt (DE)

Correspondence Address: BRINKS HOFER GILSON & LIONE, P.O. BOX 10395, CHICAGO, IL, 60610, US

(21) Appl. No.: 11/236,480

(22) Filed: Sep. 27, 2005

(30) Foreign Application Priority Data
Sep. 30, 2004 (DE) 10 2004 048 212.8

(51) Int. Cl. A61N 5/10

(52) U.S. Cl. 378/65

(57) ABSTRACT

A radiotherapy system is disclosed which provides precise, fast patient positioning. The radiotherapy system includes a gantry on which a therapy beam source that determines a beam axis and two imaging units secured rotatably about an isocentric axis. Each imaging unit includes an X-ray beam and an X-ray detector, opposite one another along respective imaging axes. The imaging axes two imaging units are oriented differently.

### FIELD

The present embodiments relate, in general, to medical systems, and in particular, to radiotherapy systems.

### BACKGROUND

Generally, the terminology “radiotherapy system” refers to a medical system where a patient is exposed for or subjected to therapeutic treatments to a high-energy photon radiation, such as an electromagnetic radiation (X-radiation, gamma radiation), or to a particle radiation (electrons, protons, carbon ions, etc.).

In the course of a radiation treatment, precise and accurate patient positioning assures that a region of a patient's body to be irradiated, such as a tumor, is exposed to a substantially high enough radiation dose, but healthy tissue of the patient is minimally damaged. For positioning, a process of locating the region to be irradiated in the patient's body is typically performed at regular time intervals. The localizing process is generally performed with imaging radiation methods, such as computed tomography. For reliably minimizing incorrect positioning of the patient, the examination may be done directly in the irradiation position.

For photon radiotherapy, a radiotherapy system is known from European Patent Disclosure EP 0 382 560 A1. A therapy beam generator which emits X-radiation is simultaneously used as part of an imaging system. An X-ray detector is located opposite the therapy beam generator in the beam direction. The X-radiation emitted by the beam generator is partly attenuated to have comparatively slight radiation intensity adequate for imaging purposes. This attenuated radiation is picked up or captured and evaluated by imaging technology for locating the body region to be irradiated. A portion of the beam that has substantially greater radiation intensity is applied as a therapeutic beam to the body region to be irradiated.

A radiological tomographic imaging system is also mounted directly on a gantry that rotatably holds a therapy beam outlet or source. The rotation of the imaging system around the patient for tomographic imaging is performed together with the gantry rotation. Because of the typically comparatively slow rotation speed of the gantry in a radiotherapy system, however, this imaging technique is comparatively time-consuming.

From US Patent Disclosure US 2003/0048868 A1, German Patent Disclosure DE 102 31 630 A1, and U.S. Pat. Nos. 6,307,914 B1 and 5,207,233 A, two radiological imaging systems can be assigned to a radiotherapy system for the sake of locating the body region to be irradiated. The respective imaging axes of these systems are oriented crosswise to one another. In U.S. Pat. No. 5,207,233 A, the imaging systems are mounted jointly on a gantry together with a linear accelerator as the therapy beam generator.

### OBJECT AND SUMMARY

The present invention is defined by the appended claims. This description summarizes some aspects of the present embodiments and should not be used to limit the claims.

A radiotherapy system with substantially precise, fast patient positioning is provided. The radiotherapy system includes a gantry, a supporting frame that is rotatable about an isocentric axis. The gantry holds a therapy beam source which determines a beam axis that is aimed at or intersects the isocentric axis. At least two imaging units may be mounted on the gantry. Each imaging unit includes one X-ray beam and one X-ray detector, opposite one another along an imaging axis. Both imaging units are rotatable about the isocentric axis by the gantry rotation. The imaging axes of two imaging units are oriented differently in a surrounding room, or relative to a patient placed or positioned in the irradiation position.

Making images from radiological projections, that is, two-dimensional radiological images for patient positioning, is accomplished during the same rotary motion predetermined by the gantry, with two or more imaging units. At each gantry position, images are taken simultaneously of radiological projections from different projection directions. The time for data acquisition is shortened considerably by using a plurality of imaging units simultaneously, improving utilization of the radiotherapy system and hence reduced treatment costs per patient.

In one aspect, to enable covering a substantially large three-dimensional volume with the imaging units, the X-ray beam detector of at least one imaging unit is disposed tangentially, eccentrically relative to the associated imaging axis. In other words, the detector is offset in or counter to the rotation direction of the gantry such that an X-ray beam, emitted along the imaging axis, strikes the detector eccentrically. The detectors of at least two imaging units may be offset contrary or opposite to one another. For instance, if the detector of the first imaging unit is offset in a rotation direction of the gantry, then the detector of the second imaging unit is offset counter to the rotation direction, or vice versa. For a comparatively large imaging volume, a comparatively fast imaging time is simultaneously achieved, since the asymmetry of the detector arrangement is compensated for by the contrary offset of the two detectors, and all of the three-dimensional information is already acquired in a gantry rotation about an angle that is substantially less than 360°.

In another aspect, the imaging axes of at least two imaging units are offset from one another axially relative to the isocentric axis. During a gantry rotation, a comparatively large axial region of the patient's body along the isocentric axis is simultaneously covered, contributing to a substantial shortening of the imaging time. The imaging axes of two imaging units may be oppositely offset relative to the X-ray beam axis, so that the X-ray beam axis is disposed between the two imaging axes in the direction of the isocentric axis.

In another embodiment, the imaging units are mounted on the gantry in such a way that each associated imaging axis extends in an image plane oriented perpendicularly to the isocentric axis. The imaging axes of two imaging units are disposed at a predetermined angular offset as viewed in projection along the isocentric axis. Two imaging units may be provided. The angular offset of the two associated imaging axes is preferably between 40° and 130°. Alternately, the angular offset of the two imaging axes is approximately 90° or approximately 60°.

In still another embodiment, the imaging axes of two imaging units are also oriented mirror-symmetrically relative to the beam axis. The therapy beam outlet is disposed between the detectors of the imaging units. In a particle radiotherapy system, the detectors of the imaging units may be mounted directly on the therapy beam outlet.

In a further embodiment, at least one of the imaging units or each imaging unit is embodied as a cone beam imaging system. This is understood to be a tomographic radiological imaging technique in which the X-ray beam of an imaging unit emits a conical beam which is picked up or received by a two-dimensional X-ray detector. Upon a rotation of the imaging unit around the patient, a volumetric region, not merely a thin tomographic slice, of the patient's body is imaged. The cone beam technique makes comparatively fast data acquisition of extended body volumes possible and is therefore substantially suitable in a radiotherapy system.

Illustrative and exemplary embodiments of the invention are described in further detail below with reference to, and in conjunction with, the figures.

### BRIEF DESCRIPTION OF THE DRAWINGS

FIG. 1 is a schematic top view along an isocentric axis, and shows a gantry of a radiotherapy system with a therapy beam outlet and with two radiological tomographic imaging units for patient positioning;

FIG. 2 is a schematic view along the line II-II of FIG. 1, showing a side view the gantry of FIG. 1; and

FIG. 3 is a schematic view as that of FIG. 2, showing an alternate example of the gantry of FIG. 1, in which two imaging units are offset axially from one another relative to an isocentric axis.

Similar parts and elements are identified by the same reference numerals or symbols in all the drawings.

### DETAILED DESCRIPTION

The above, as well as other, advantages of the present invention will become readily apparent to those skilled in the art from the following detailed description of preferred embodiments when considered in the light of the accompanying drawings.

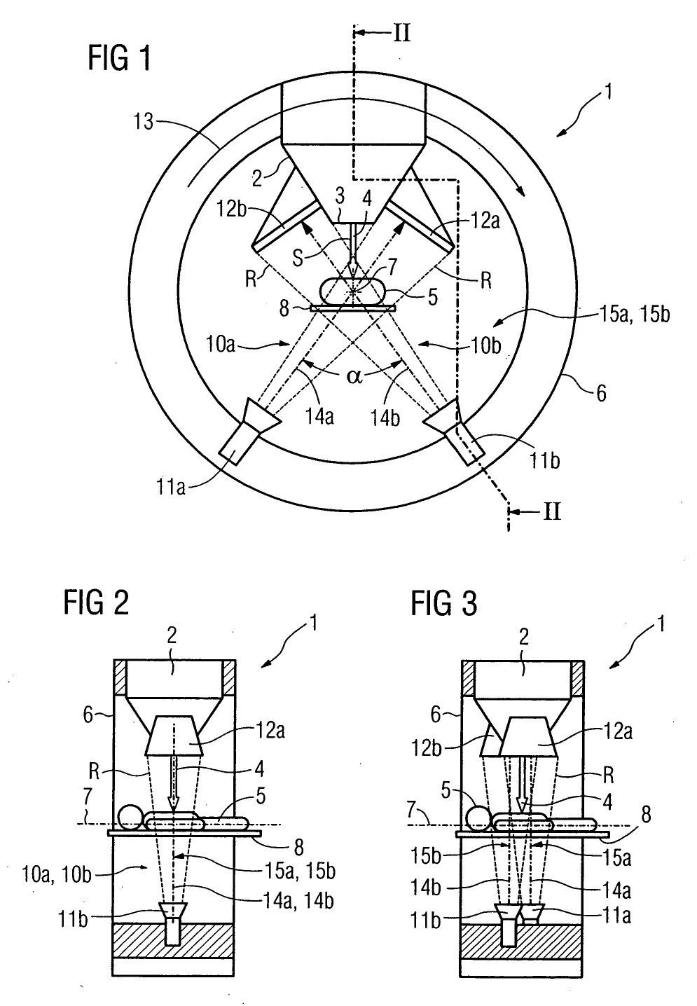

FIG. 1 shows a schematic end view of a radiotherapy system 1, hereinafter called system 1 for short. The system 1 includes a therapy beam outlet or source 2, and an orifice 3 from which a therapy or treatment beam S is emitted along a beam axis 4 toward a body of a patient 5. The system 1 may be a system for particle beam therapy. The therapy beam source 2 is connected here to a particle accelerator (not shown). The therapy beam S may contain particles, such as protons, carbon ions, etc., that are accelerated to a high speed.

The therapy beam source 2 is mounted on a gantry 6 which has a substantially annular supporting frame. The gantry 6 is rotatable about an isocentric axis 7. By rotation of the gantry 6, the therapy beam source 2 and thus the beam axis 4 are pivotable about the patient 5 placed or positioned on a patient table 8 approximately in the region of an isocentric axis 7.

In order to locate the body region of the patient 5 to be irradiated and, on the basis of this location, to enable positioning the patient 5 suitably, the system 1 further includes two imaging units 10a and 10b. Each imaging unit 10a and 10b includes a respective X-ray beam 11a and 11b and a respective digital X-ray beam detector 12a and 12b. The X-ray detectors 12a and 12b are mounted opposite one another on the therapy beam source 2, so that the X-ray detector 12a proceeds the therapy beam outlet 2 in a rotation direction 13 of the gantry 6, and the X-ray detector 12b follows the therapy beam outlet 2 in the rotation direction 13. The X-ray beam sources 11a and 11b are mounted on a side of the gantry 6 opposite the therapy beam source 2. The X-ray beam sources 11a and 11b of each imaging unit 10a, 10b is located opposite, along an associated imaging axis 14a and 14b, from the respective associated X-ray detectors 12a and 12b. The imaging axes 14a and 14b are each determined by central beams of the X-radiation R emitted by the respective X-ray beam sources 11a and 11b.

The imaging units 10a, 10b may be cone beam imaging systems. The X-radiation R emitted by each of the X-ray beams sources 11a, 11b has a conical emission characteristic. In other words, the beam originating from each X-ray beam source 11a, 11b broadens with increasing distance, both within an image plane 15a, 15b extending perpendicular to the isocentric axis 7 and in the direction perpendicular to that plane.

The imaging units 10a, 10b are positioned inside the gantry 6 so the respective imaging axes 14a, 14b are aimed essentially radially relative to the isocentric axis 7 and thus intersect that axis at approximately a right angle. Each imaging axis 14a, 14b extends within the associated image plane 15a, 15b. The imaging units 10a, 10b may be located at the same height relative to the isocentric axis 7, so that the image planes 15a and 15b coincide (FIG. 2). Alternately, the imaging units 10a, 10b may be conversely offset axially from one another as shown in FIG. 3, so that two parallel, spaced-apart image planes 15a and 15b are determined by the imaging axes 14a and 14b. In FIG. 3, the imaging units 10a and 10b are offset in opposite axial directions relative to the beam axis 4, so that the beam axis 4 is located approximately centrally between the image planes 15a and 15b. The imaging volume, that is, the three-dimensional region scanned by imaging technology by the imaging units 10a and 10b in the axial region is enlarged. The imaging units 10a and 10b may be disposed such that the imaging volumes of the two imaging units 10a and 10b overlap in the region of the beam axis 4.

As shown in FIG. 1, the imaging units 10a, 10b are aimed or oriented such that their respective imaging axes 14a and 14b intersect at an offset angle a in projection along the isocentric axis 7. The offset angle α is, for structural regions, approximately 60°. Alternately, the offset angle is approximately 90° or other angle (not shown).

As shown in FIG. 1, the X-ray detectors 12a and 12b may not be disposed centrally relative to the respective imaging axis 14a and 14b, but rather tangentially eccentrically. This eccentricity may be pronounced or emphasized in opposite directions for the two imaging units 10a and 10b. The X-ray detector 12a of the imaging unit 10a is thus offset relative to a central position in the rotation direction 13, while the X-ray detector 12b of the imaging unit 10b is offset counter to the rotation direction 13. Due to this tangential eccentric disposition of the X-ray detectors 12a and 12b, an increase in the imaging volume in the radial direction is provided, as compared to a central arrangement, for a given detector size. The opposite offset of the two imaging units 10a and 10b compensates for an asymmetry of the data acquisition brought about by the eccentric detector arrangement. Upon a one-half gantry rotation, substantially the entire volumetric information may be an available.

For locating the region to be irradiated of the body of the patient 5, two-dimensional X-ray images are taken by the imaging units 10a and 10b from many projection directions. From these images, a digital evaluation unit (not shown) computes a three-dimensional image data set for the examined body region of the patient 5. The imaging is performed, as typical in radiological tomographic images, by rotation of the gantry 6 and the imaging units 10a and 10b mounted on or secured to the gantry 6 around the patient 5. The imaging is performed before an irradiation phase begins, to enable calibrating the body region to be irradiated of the patient 5 with the details of the image information in the isocenter of the therapy beam S. The imaging can also be continued during the irradiation phase, for monitoring purposes.

By simultaneously using two imaging units 10a and 10b, a substantial reduction in imaging time may be achieved. Such simultaneous imaging may also reduces the total duration of the radiation session, that is, the length of time that the patient 5 needs to be placed or positioned in the irradiation position inside the gantry 6. Because of the shortened treatment time, a higher patient throughput in the system 1 can be achieved, which represents a decisive advantage, from the standpoint of the high operating costs for a radiotherapy system.

1. A radiotherapy system with a gantry, the radiotherapy system comprising:
a therapy beam source defining a beam axis;
a first imaging unit comprising a first X-ray beam and a first X-ray detector opposite one another along a first imaging axis; and
a second imaging unit comprising a second X-ray beam and a second X-ray detector opposite one another along a second imaging axis, wherein the first and second imaging axes are oriented differently, and at least one of the first and second X-ray detectors is disposed tangentially eccentrically relative to the associated imaging axis;
wherein the therapy beam source, the first and second imaging units are mounted rotatably about an isocentric axis.

2. The radiotherapy system according to claim 1, wherein the first and second X-ray detectors are disposed oppositely eccentrically to one another relative to the beam axis.

3. The radiotherapy system according to claim 1, wherein the first and second imaging units and the therapy beam source are mounted on the gantry.

4. A radiotherapy system having a gantry, the radiotherapy system comprising:
a therapy beam source defining a beam axis;
a first imaging unit comprising a first X-ray beam and a first X-ray detector opposite one another along a first imaging axis; and
a second imaging unit comprising a second X-ray beam and a second X-ray detector opposite one another along a second imaging axis;
wherein the therapy beam source, the first imaging unit and the second imaging unit are mounted rotatably about an isocentric axis, the first and second imaging axes are oriented differently, and are disposed offset from one another axially relative to the isocentric axis.

5. The radiotherapy system according to claim 4, wherein the first and second imaging axes are oppositely offset relative to the beam axis.

6. The radiotherapy system according to claim 1, wherein each of the first and second imaging axes extends in a corresponding image plane perpendicular to the isocentric axis, and the first imaging axis is disposed at a predetermined angular offset (α), in projection along the isocentric axis, with respect to the second imaging axis.

7. The radiotherapy system according to claim 6, wherein the angular offset (α) is between 40° and 130°.

8. The radiotherapy system according to one of claim 6, wherein the first and second imaging axes are oriented mirror-symmetrically, in projection along the isocentric axis, relative to the beam axis.

9. The radiotherapy system according to claim 1, wherein at least one of the first and second imaging units comprises a cone beam imaging system.

10. The radiotherapy system according to claim 1, wherein at least one of the first and second X-ray detectors is mounted on the therapy beam source.

11. The radiotherapy system according to claim 3, wherein each of the first and second imaging axes extends in a corresponding image plane perpendicular to the isocentric axis, and the first imaging axis is disposed at a predetermined angular offset (α), in projection along the isocentric axis, with respect to the second imaging axis.

12. The radiotherapy system according to claim 11, wherein the angular offset (α) is between 40° and 130°.

13. The radiotherapy system according to one of claim 11, wherein the first and second imaging axes are oriented mirror-symmetrically, in projection along the isocentric axis, relative to the beam axis.

14. The radiotherapy system according to claim 3, wherein at least one of the first and second imaging units comprises a cone beam imaging system.

15. The radiotherapy system according to claim 3, wherein at least one of the first and second X-ray detectors is mounted on the therapy beam source.

16. The radiotherapy system according to claim 7, wherein at least one of the first and second imaging units comprises a cone beam imaging system.

17. The radiotherapy system according to claim 1 1, wherein at least one of the first and second imaging units comprises a cone beam imaging system.
